(19) United States
(12) Patent Application Publication
(10) Pub. No.: US 2025/0325255 A1
(43) Pub. Date: Oct. 23, 2025

(54) CONTRAST-ENHANCED ULTRASOUND IMAGING METHOD AND ULTRASOUND IMAGING APPARATUS

(71) Applicant: Shenzhen Mindray Bio-Medical Electronics Co., Ltd., Shenzhen (CN)

(72) Inventors: Yali OUYANG, Shenzhen (CN); Maodong SANG, Shenzhen (CN); Lei ZHU, Shenzhen (CN); Xujin HE, Shenzhen (CN)

(21) Appl. No.: 19/253,585

(22) Filed: Jun. 27, 2025

Related U.S. Application Data
(63) Continuation of application No. PCT/CN2023/141194, filed on Dec. 22, 2023.

(51) Int. Cl. A61B 8/00

(52) U.S. Cl. CPC A61B 8/54; A61B 8/481

(57) ABSTRACT

Discloses are a CEUS imaging method and an ultrasound imaging apparatus. The method includes: controlling an ultrasound probe to transmit multiple ultrasound pulse combinations to a target object injected with a contrast agent and receive ultrasound echo signals, wherein each combination comprises multiple consecutive single pulses and one pulse sequence, the single pulses are of the same amplitude, and the pulse sequence comprises at least two pulses with different amplitudes; acquiring first echo signals corresponding to the pulse sequence from the ultrasound echo signals, generating and real-time displaying a contrast microbubble image based on the first echo signals; acquiring second echo signals corresponding to the single pulses from the ultrasound echo signals, and generating and displaying a SR-CEUS image based on the second echo signals. This method enables ultra-high frame rate data acquisition to reduce super-resolution imaging data collection time, while concurrently realizing real-time visualization of microbubble dynamics.

### CROSS-REFERENCE TO RELATED APPLICATIONS

This application is a bypass continuation of Patent Cooperation Treaty Application No. PCT/CN2023/141194, entitled “CONTRAST-ENHANCED ULTRASOUND IMAGING METHOD AND ULTRASOUND IMAGING APPARATUS” filed on Dec. 22, 2023, which claims priority to Chinese Patent Application No. 202211700266.8, entitled “CONTRAST-ENHANCED ULTRASOUND IMAGING METHOD AND ULTRASOUND IMAGING APPARATUS” filed on Dec. 28, 2022, both of which are incorporated herein by reference in their entirety.

### TECHNICAL FIELD

The present disclosure relates to the technical field of ultrasound imaging, and more particularly to contrast-enhanced ultrasound imaging methods and ultrasound imaging apparatus.

### BACKGROUND OF THE DISCLOSURE

Contrast-enhanced ultrasound (CEUS), as a new technique capable of real-time dynamic observation of lesions and their tissue blood perfusion, plays an increasingly important role in the diagnosis of malignant diseases such as liver cancer, thyroid cancer, and breast cancer, and has become an essential examination method for clinical evaluation of blood circulation and perfusion.

The obstruction, blockage, and pathological changes in microcirculation are precursors to many diseases. Observing microvascular changes facilitates early disease diagnosis. Capillaries, located in the epidermal layer, constitute a critical component of blood microcirculation with minimal luminal diameters (approximately 6-9 μm). Arterioles and venules reside in the dermal layer, measuring 10-100 μm in diameter, connecting to arteries and veins in the hypodermal layer. Microcirculation refers to the blood flow between arterioles and venules within the vascular network, serving both as the terminal portion of the circulatory system and an essential constituent of visceral organs. Normally, microcirculatory blood flow adapts to the metabolic demands of human tissues and organs, sustaining normal vital activities and metabolism. When tissue/organ metabolism and function become abnormal, microcirculation undergoes specific alterations. Consequently, microcirculation is intrinsically linked to disease initiation and progression, holding significant physiological, pathological, pharmacological, and clinical implications that prove invaluable for early diagnosis and treatment of various diseases.

However, due to the diffraction limit constraints of ultrasound in the far field, conventional clinical CEUS exhibits limited capability in displaying microvascular structural details. Although spatial resolution can be improved by increasing transmission frequency and implementing near-field imaging, this inevitably leads to reduced imaging depth. Given that most organs are located at substantial depths from the probe, near-field super-resolution methods prove difficult to apply. Super-resolution contrast-enhanced ultrasound (SR-CEUS), an emerging imaging method with ultra-high spatial resolution, achieves images at a scale of tens of micrometers by adapting principles from fluorescence microscopy localization techniques in optical super-resolution imaging, specifically through the localization and tracking of isolated microbubbles. Consequently, SR-CEUS resolves the challenge of microvascular visualization, emerging as a powerful tool for observing micro-blood flow. It is currently widely applied in preclinical studies across fields including tumor imaging, microhemodynamic perfusion in various organs, and neovascularization within plaques. The current SR-CEUS transmission strategy employs conventional frame-rate pulse sequences, acquiring raw image data at standard frame rates. This approach requires prolonged acquisition times to obtain sufficient raw image data for super-resolution processing, resulting in suboptimal imaging efficiency.

### SUMMARY OF THE DISCLOSURE

A contrast-enhanced ultrasound imaging method provided according to a first aspect of the present disclosure may include:

controlling an ultrasound probe to transmit a plurality of ultrasound pulse combinations to a target object injected with a contrast agent and receive ultrasound echo signals, wherein each of the ultrasound pulse combinations comprises a plurality of consecutive single pulses and one pulse sequence, the single pulses are of the same amplitude, and the pulse sequence comprises at least two pulses with different amplitudes;
acquiring first echo signals corresponding to the pulse sequence from the ultrasound echo signals, generating and real-time displaying a contrast microbubble image based on the first echo signals; and
acquiring second echo signals corresponding to the single pulses from the ultrasound echo signals, and generating and displaying a SR-CEUS image based on the second echo signals.

A contrast-enhanced ultrasound imaging method provided according to a second aspect of the present disclosure may include:
controlling an ultrasound probe to transmit an ultrasound pulse combination to a target object injected with a contrast agent, and receive ultrasound echo signals, wherein the ultrasound pulse combination comprises at least a first pulse sequence and at least a second pulse sequence, the first pulse sequence comprises one single pulse and/or a plurality of consecutive single pulses, the single pulses are of the same amplitude, and the second pulse sequence comprises at least two pulses with different amplitudes;
acquiring first echo signals corresponding to the second pulse sequence from the ultrasound echo signals, generating and real-time displaying a contrast microbubble image based on the first echo signals; and
acquiring second echo signals corresponding to the single pulses from the ultrasound echo signals, and generating and display a SR-CEUS image based on the second echo signals.

A contrast-enhanced ultrasound imaging method provided according to a third aspect of the present disclosure may include:
controlling an ultrasound probe to transmit a plurality of ultrasound pulse combinations to a target object injected with a contrast agent, and receive ultrasound echo signals, wherein each of the ultrasound pulse combinations comprises a plurality of consecutive single pulses and one pulse sequence, the single pulses are of the same amplitude, the pulse sequence comprises at least two pulses with different amplitudes, and at least one pulse in the pulse sequence has an amplitude equal to the amplitude of the single pulses;
acquiring first echo signals corresponding to the pulse sequence from the ultrasound echo signals, generating and real-time displaying a contrast microbubble image based on the first echo signals; and
acquiring, from the ultrasound echo signals, second echo signals corresponding to the single pulses and third echo signals corresponding to the pulses in the pulse sequence that have the same amplitude as the single pulses, and generating and displaying a SR-CEUS image based on the second echo signals and the third echo signals.

A contrast-enhanced ultrasound imaging method provided according to a fourth aspect of the present disclosure may include:
controlling an ultrasound probe to transmit an ultrasound pulse combination to a target object injected with a contrast agent and receive ultrasound echo signals, wherein the ultrasound pulse combination comprises at least a first pulse sequence and at least a second pulse sequence, the first pulse sequence comprises one single pulse and/or a plurality of consecutive single pulses, the single pulses are of the same amplitude, the second pulse sequence comprises at least two pulses with different amplitudes, and the amplitude of at least one pulse in the second pulse sequence is identical to the amplitude of the single pulses;
acquiring first echo signals corresponding to the pulse sequence from the ultrasound echo signals, generating and real-time displaying a contrast microbubble image based on the first echo signals; and
acquiring, from the ultrasound echo signals, second echo signals corresponding to the single pulses and third echo signals corresponding to the pulses in the second pulse sequence that have the same amplitude as the single pulses, generating and displaying a SR-CEUS image based on the second echo signals and the third echo signals.

A contrast-enhanced ultrasound imaging method provided according to a fifth aspect of the present disclosure may include:
controlling an ultrasound probe to transmit a plurality of ultrasound pulse combinations to a target object injected with a contrast agent and receive ultrasound echo signals, wherein each of the ultrasound pulse combination comprises a plurality of consecutive single pulses and one pulse sequence, the single pulses are of the same amplitude, and the pulse sequence comprises at least two pulses with different amplitudes;
acquiring first echo signals corresponding to the pulse sequence from the ultrasound echo signals, generating and real-time displaying a contrast microbubble image based on the first echo signals; and
generating and displaying a SR-CEUS image based on the ultrasound echo signals.

A contrast-enhanced ultrasound imaging method provided according to a sixth aspect of the present disclosure may include:
controlling an ultrasound probe to transmit an ultrasound pulse combination to a target object injected with a contrast agent and receive ultrasound echo signals, wherein the ultrasound pulse combination comprises at least a first pulse sequence and at least a second pulse sequence, the first pulse sequence comprises one single pulse and/or a plurality of consecutive single pulses, the single pulses of the same amplitude, and the second pulse sequence comprises at least two pulses with different amplitudes;
acquiring first echo signals corresponding to the second pulse sequence from the ultrasound echo signals, generating and real-time displaying a contrast microbubble image based on the first echo signals; and
generating and displaying a SR-CEUS image based on the ultrasound echo signals.

A contrast-enhanced ultrasound imaging method provided according to a seventh of the present disclosure may include:
controlling an ultrasound probe to transmit one or more ultrasound pulse sequences to a target object injected with a contrast agent and receive first echo signals wherein the one or more ultrasound pulse sequences comprise at least two pulses with different amplitudes;
generating and displaying a contrast microbubble image based on the first echo signals, and acquiring a region of interest (ROI) of the target object based on the contrast microbubble image;
controlling the ultrasound probe to transmit a plurality of ultrasound single pulses to the ROI of the target object and receive second echo signals, wherein the single pulses are of the same amplitude; and
generating and displaying a SR-CEUS image based on the second echo signals.

An ultrasound imaging apparatus provided according to an eighth of the present disclosure may include: a transmit-receive circuit, an ultrasound probe, a processor, and a display; wherein
the transmit-receive circuit is configured to control the ultrasound probe to transmit ultrasound waves to a target object and receive echoes of the ultrasound waves, and acquire ultrasound echo signals from the echoes;
the processor is configured to control the transmit-receive circuit and execute the above contrast-enhanced ultrasound imaging method to generate a contrast microbubble image and a SR-CEUS image; and
the display is configured to display the contrast microbubble image and the SR-CEUS image.

By transmitting an ultrasound pulse combination comprising a first pulse sequence having one or more single pulses of the same amplitude and a second pulse sequence having at least two pulses with different amplitudes to a target object injected with a contrast agent, utilizing echo signals of the first pulse sequence for SR-CEUS imaging, and utilizing echo signals of the second pulse sequence for real-time microbubble imaging, the CEUS imaging method and the ultrasound imaging apparatus disclosed according to the present disclosure can achieve ultra-high frame rate data acquisition to reduce super-resolution imaging data collection time while concurrently realizing real-time visualization of microbubble dynamics. Consequently, doctors can not only monitor microbubble perfusion status but also obtain SR-CEUS images to examine microvascular structures and other minute tissue details.

### BRIEF DESCRIPTION OF THE DRAWINGS

By providing a more detailed description of the embodiments of the present disclosure with reference to the accompanying drawings, the aforementioned objectives, features, and advantages of the present disclosure, as well as others, will become more apparent. The drawings are intended to enhance the understanding of the embodiments of the present disclosure and form part of the specification. Together with the embodiments of the present disclosure, they serve to explain the disclosure and do not constitute a limitation on the present disclosure. In the drawings, identical reference numerals typically represent identical components or steps.

FIG. 1 shows a schematic flowchart of a contrast-enhanced ultrasound (CEUS) imaging method according to some embodiments of the present disclosure;

FIG. 2 shows a more specific exemplary flowchart of a CEUS method according to some embodiments of the present disclosure;

FIG. 3 shows a schematic diagram of transmissions of pulse sequences in a CEUS method according to some embodiments of the present disclosure;

FIG. 4 schematically illustrates pulse responses of linear and nonlinear media;

FIG. 5 shows a schematic diagram of a transmission strategy in a CEUS method according to some embodiments of the present disclosure;

FIG. 6 shows schematic diagram of another transmission strategy in a CEUS method according to some embodiments of the present disclosure;

FIG. 7 shows a schematic flowchart of a CEUS method according to other embodiments of the present disclosure;

FIG. 8 shows a schematic flowchart of a CEUS method according to other embodiments of the present disclosure;

FIG. 9 shows a schematic flowchart of a CEUS method according to other embodiments of the present disclosure;

FIG. 10 shows a schematic flowchart of a CEUS method according to other embodiments of the present disclosure;

FIG. 11 shows a schematic flowchart of a CEUS method according to other embodiments of the present disclosure;

FIG. 12 shows a schematic flowchart of a CEUS method according to other embodiments of the present disclosure; and

FIG. 13 shows a schematic structural block diagram of an ultrasound imaging apparatus according to some embodiments of the present disclosure.

### DESCRIPTION OF THE PREFERRED EMBODIMENTS

To make the objectives, technical solutions, and advantages of the present disclosure more apparent, the following describes exemplary embodiments of the present disclosure in detail with reference to the accompanying drawings. It is evident that the described embodiments represent only a portion of the embodiments of the present disclosure and not all possible embodiments. It should be understood that the present disclosure is not limited by the exemplary embodiments described herein. Based on the embodiments of the present disclosure disclosed herein, all other embodiments obtained by those skilled in the art without creative efforts shall fall within the protection scope of the present disclosure.

In current super-resolution imaging scenarios, conventional frame-rate pulse sequences are employed for raw image data acquisition, where the low frame rate necessitates prolonged acquisition times to obtain sufficient raw image data for super-resolution processing. While increasing ultrasound frame rates can reduce acquisition time by enabling rapid collection of adequate raw data, excessively high frame rates prevent real-time processing and visualization of microbubble dynamics during acquisition. The solution as disclosed herein achieves concurrent ultra-high frame rate data acquisition (thereby reducing super-resolution imaging data collection time) and real-time display of microbubble dynamics. This dual capability allows doctors to not only monitor microbubble perfusion status in real time but also obtain super-resolution images for observing microvascular structures and other microscopic tissue details. The specific implementations of this application are described as follows.

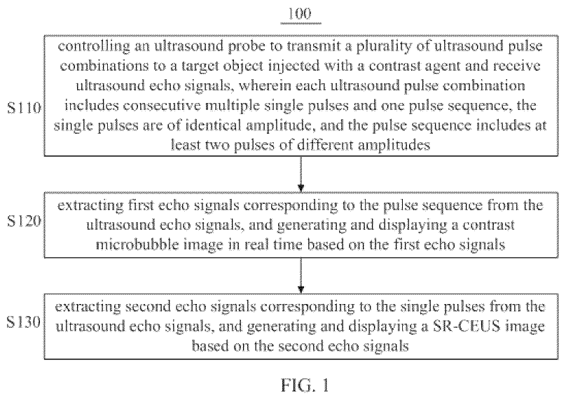

FIG. 1 illustrates a schematic flowchart of a contrast-enhanced ultrasound imaging method (CEUS) method 100 according to some embodiments of the present disclosure. As shown in FIG. 1, the CEUS method 100 may comprise the following steps:

Step S110: controlling an ultrasound probe to transmit a plurality of ultrasound pulse combinations to a target object injected with a contrast agent and receive ultrasound echo signals, wherein each of the ultrasound pulse combinations includes a plurality of consecutive single pulses and one pulse sequence, the single pulses are of identical amplitude, and the pulse sequence includes at least two pulses of different amplitudes.

Step S120: extracting first echo signals corresponding to the pulse sequence from the ultrasound echo signals, and generating and real-time displaying a contrast microbubble image based on the first echo signals.

Step S130: extracting second echo signals corresponding to the single pulses from the ultrasound echo signals, and generating and displaying a super-resolution contrast-enhanced ultrasound (SR-CEUS) image based on the second echo signals.

In embodiments of the present disclosure, an ultrasound probe is controlled to transmit a plurality of ultrasound pulse combinations to a target object injected with a contrast agent, and CEUS imaging is performed based on the received ultrasound echo signals. Specifically, each ultrasound pulse combination includes a plurality of consecutive single pulses and one pulse sequence. A single pulse, as the name implies, refers to one pulse, while the pulse sequence contains at least two pulses. More specifically, the plurality of consecutive single pulses in each ultrasound pulse combination have the same amplitude (i.e., uniform single pulses), and the one pulse sequence in each ultrasound pulse combination includes at least two pulses with different amplitudes. Based on such transmissions, the receiving end correspondingly receives ultrasound echo signals. For differentiation: echo signals corresponding to the pulse sequence are referred to as first echo signals; echo signals corresponding to the single pulses are referred to as second echo signals; and the ultrasound echo signals collectively include all echo signals (both the first and second echo signals). In this disclosure, the first echo signals corresponding to the pulse sequence are extracted from the ultrasound echo signals to generate and display a contrast microbubble image in real time, while the second echo signals corresponding to the single pulses are extracted from the ultrasound echo signals to generate and display a SR-CEUS image. This transmission, reception and processing strategy is adopted because: (i) under single-angle transmission: a single pulse (containing one pulse) transmits one pulse to the same position of the target object, while a pulse sequence (containing at least two pulses) transmits at least two pulses to the same position; (ii) under multi-angle transmission: each single pulse is transmitted multiple times from multiple angles to the same position, and each pulse in the pulse sequence is also transmitted multiple times from multiple angles to the same position, and accordingly, the total number of transmissions to the same position exceeds that of single-pulse transmissions. Conversely, excessively high frame rates may compromise real-time processing and display of microbubble dynamics. To address this, the present disclosure interleaves conventional-contrast-compatible pulse sequences (where each ultrasonic pulse combination includes one pulse sequence) within high-frame-rate single pulses. The echo signals corresponding to these pulse sequences can be extracted for real-time microbubble imaging.

In an example, one ultrasound pulse combination is transmitted per second, each comprising 50 single pulses and 1 pulse sequence. In another example, 500 to 2500 single pulses and 1 to 20 pulse sequences are transmitted per second. Other examples may involve different quantities of single pulses and pulse sequences transmitted per second. The number of single pulses per ultrasound pulse combination, or the quantities of single pulses and pulse sequences transmitted per second, are determined as needed. Generally, the higher the resolution requirement for super-resolution images, the greater the number of single pulses and the fewer the number of interleaved pulse sequences in the overall transmission strategy. The quantity of pulse sequences primarily depends on the users' need to observe real-time microbubble dynamics; however, excessive interleaving of pulse sequences may degrade super-resolution imaging performance. Consequently, a trade-off should be made based on specific requirements to configure the number of single pulses per ultrasonic pulse combination or the quantities of single pulses and pulse sequences transmitted per second.

In summary, the contrast-enhanced ultrasound imaging method 100 disclosed herein transmits ultrasonic pulse combinations comprising one or more single pulses and one or more pulse sequences to a target object injected with a contrast agent, and utilizes echo signals corresponding to the single pulses for SR-CEUS imaging and echo signals corresponding to the pulse sequences for real-time microbubble imaging, thereby simultaneously achieving ultra-high-frame-rate data acquisition to reduce super-resolution imaging data collection time while enabling real-time visualization of microbubble dynamics. This allows doctors to observe both microbubble activity and acquire super-resolution images for evaluating microvascular and other fine tissue structures.

In embodiments of the present disclosure, the amplitude of the aforementioned single pulses is a first amplitude, and the amplitude of at least one pulse in the pulse sequence is the first amplitude. This first amplitude is generally greater than the amplitude corresponding to a half-amplitude pulse and less than or equal to the amplitude corresponding to a full-amplitude pulse. For example, the amplitude of the single pulse may be the amplitude corresponding to a full-amplitude pulse, and the amplitude of at least one pulse in the pulse sequence is the amplitude corresponding to a full-amplitude pulse. In one example, the pulse sequence may include three pulses, where the amplitude of one pulse equals the sum of the amplitudes of the other two pulses. For instance, within the pulse sequence, one pulse may have an amplitude corresponding to a full-amplitude pulse, while the remaining two pulses may have amplitudes represented as a (where 0<a<1) and 1−a, respectively. This pulse sequence may thus be represented as (a, 1, 1−a). The phase of the pulse sequence may be modified; for example, the pulse sequence could be (a, −1, 1−a). In addition, the order of pulses within the pulse sequence may also be adjusted, such as (1, a, 1−a), (1−a, a, 1), or (1−a, 1, a). Additionally, the number of pulses in the pulse sequence may vary as long as it includes at least two. In another example, the pulse sequence may include two pulses, where one pulse has the amplitude corresponding to a full-amplitude pulse, and the other pulse has the amplitude corresponding to a half-amplitude pulse. The pulse sequence in this example can be expressed as (0.5, 1) or (1, 0.5). In yet another example, the pulse sequence may include N pulses (where N≥2), such as (a, 1, 1, . . . , 1, 1−a), containing N−2 full-amplitude pulses; and non-full-amplitude pulses may be positioned anywhere within the pulse sequence. In other examples, the amplitudes of the pulses may exhibit symmetric distributions. For instance, in the aforementioned (a, 1, 1−a) configuration, when a=0.5, the pulse sequence becomes (0.5, 1, 0.5). This serves as one illustrative case; other symmetric configurations are also applicable but not exhaustively listed here.

In embodiments of the present disclosure, prior to controlling the ultrasound probe to transmit multiple ultrasound pulse combinations to the target object injected with the contrast agent, the method 100 may further include the following steps: controlling the ultrasound probe to transmit one or more ultrasound pulse sequences to the target object and receive third echo signals; generating and displaying a third CEUS image based on the third echo signals; determining a region of interest (ROI) of the target object based on the third contrast image; and controlling the ultrasound probe to transmit multiple ultrasound pulse combinations to the ROI of the target object. In this embodiment, transmitting the one or more ultrasound pulse sequences before Step S110 to generate the third contrast image serves to define the ROI, i.e., to identify a target imaging plane for SR-CEUS imaging. The implementation may include: displaying the third CEUS image for manual ROI selection by users via an input unit; or, automated ROI detection without image display, using algorithms such as target recognition; or, a hybrid approach combining both the manual and automated methods. The ultrasound pulse sequences used in this step may be either identical to or distinct from the pulse sequences described in the aforementioned ultrasound pulse combinations.

In embodiments of the present disclosure, an imaging region corresponding to the first echo signals and an imaging region corresponding to second echo signals are identical. In such embodiments, the real-time microbubble images generated from the first echo signals and the SR-CEUS images generated from the second echo signals correspond to the same imaging region, further enabling doctors to observe both microbubble dynamics and super-resolution vascular details within the identical region, thereby facilitating cross-referencing between the two imaging modalities. Similar to previous embodiments, in this embodiment, prior to Step S110, the method may further include: controlling the ultrasound probe to transmit one or more ultrasound pulse sequences to the target object and receive third echo signals, wherein the imaging region corresponding to the third echo signals matches that of the first or second echo signals; and generating and displaying a third CEUS image based on the third echo signals. This workflow, which performs conventional CEUS imaging before SR-CEUS imaging, allows users to confirm the spatial location of the imaging region, and assess baseline microbubble perfusion characteristics. Such preliminary information enhances the clinical utility of subsequent observations, including: real-time tracking of microbubble dynamics during SR-CEUS image imaging, and diagnostic interpretation of the final SR-CEUS images.

In embodiments of the present disclosure, the SR-CEUS image may also be generated based on both the first echo signals and the second echo signals, which can further increase the data volume available for SR-CEUS imaging and improve the quality of the SR-CEUS image.

The following describes the contrast-enhanced ultrasound imaging method 100 according to embodiments of the present disclosure with reference to specific examples and workflow implementations.

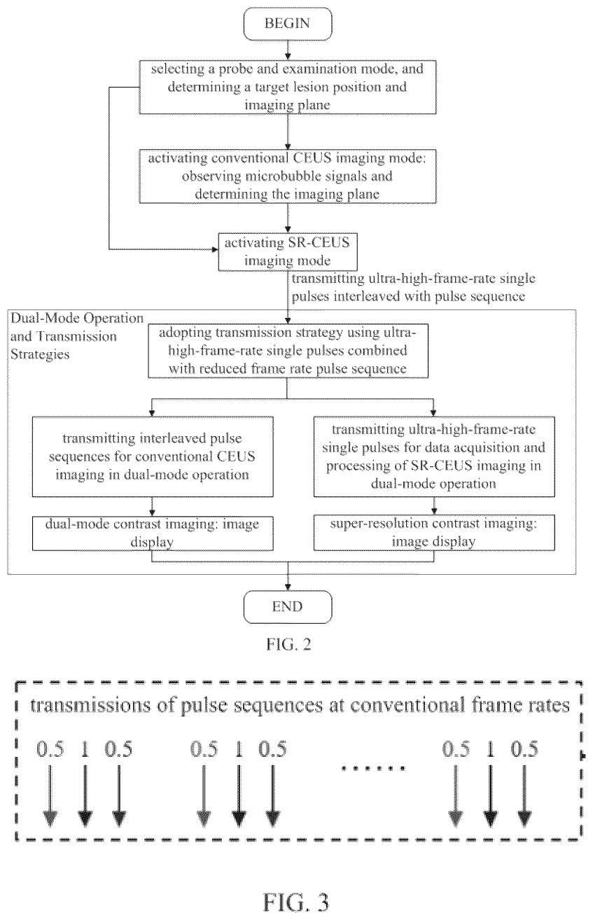

FIG. 2 illustrates a more detailed exemplary flowchart of the CEUS method according to some embodiments of the present disclosure. As shown in FIG. 2, after initiating the workflow, it may allow users to: select a probe and examination mode; determine a target lesion; and activate the conventional CEUS imaging mode of the apparatus (e.g. the steps as described above which can be executed before the Step S110), followed by activating the SR-CEUS mode (alternatively, the super-resolution contrast-enhanced ultrasound mode, abbreviated as SR-CEUS, may be directly activated, corresponding to Steps S110 to S130 as previously described). In the conventional contrast imaging mode, a conventional frame-rate pulse sequence transmission strategy is employed; and after contrast agent injection, real-time microbubble flow dynamics can be observed to define the target imaging plane.

The conventional contrast pulse sequences may employ a power/amplitude-modulated multi-pulse imaging method, such as transmitting three ultrasound pulses at the same location: first transmitting a lower-amplitude pulse (e.g., half amplitude represented as 0.5), then transmitting a higher-amplitude pulse (e.g., full amplitude represented as 1), and finally transmitting another lower-amplitude pulse (e.g., half amplitude represented as 0.5). This process is repeated at the next emission location, wherein the emission sequence of the aforementioned pulse sequence is illustrated in FIG. 3.

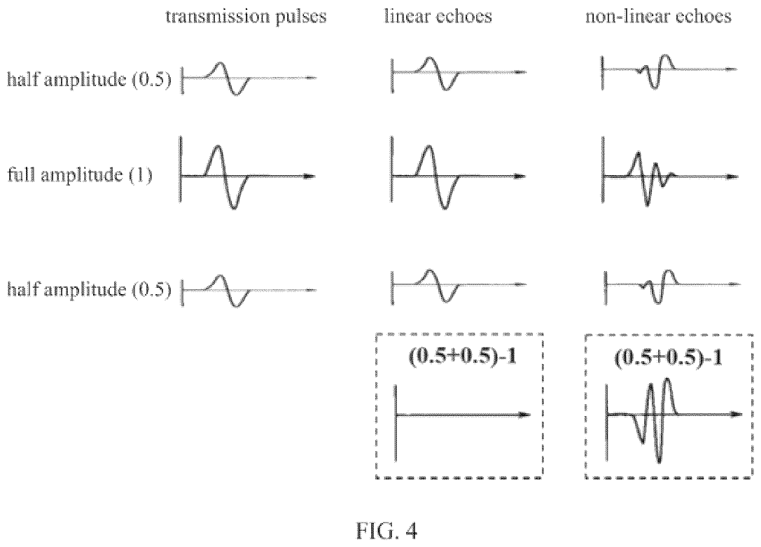

For a linear medium, the echo response signal of the second full-amplitude pulse is the sum of the first and third pulse responses, resulting in a difference of zero between them. For a nonlinear medium, the response of the second pulse is not the sum of the first and third pulse responses, and the difference between the two responses is non-zero, with the magnitude of the difference dependent on the non-linearity of the medium. Thus, after receiving the echo signals, the two low-amplitude echo signals are summed and then subtracted from the high-amplitude echo signal to extract the nonlinear component of the contrast-enhanced signal. For linear scattering, the summed superposition of linear echoes equals zero; for nonlinear scattering, the summed superposition is non-zero, thereby enhancing the nonlinear portion of the echoes. This schematic representation is illustrated in FIG. 4.

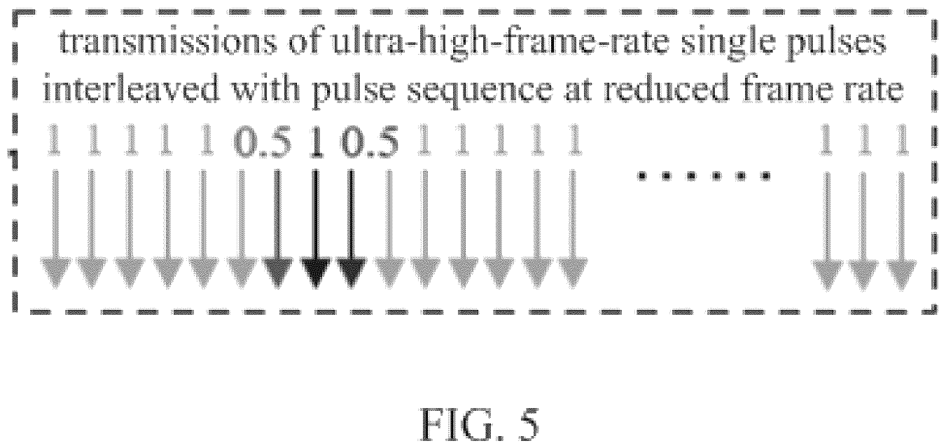

Upon initiating SR-CEUS imaging, the system enters a duplex imaging mode. Under this duplex imaging mode, an interleaved transmission strategy is employed, alternating between single pulses and pulse sequences. As illustrated in FIG. 5, this involves continuously transmitting multiple full-amplitude single pulses ([1], [1], [1], . . . ) interleaved with pulse sequences (e.g., [0.5, 1, 0.5], followed by [1], [1], [1], . . . , and another [0.5, 1, 0.5], etc.); that is, ([1], [1], [1], . . . , [0.5 1 0.5], [1], [1], [1] . . . , [0.5, 1, 0.5], [1], [1], [1] . . . ). This approach simultaneously meets the ultra-high frame rate data acquisition requirements for SR-CEUS imaging and enables real-time detection and extraction of nonlinear contrast signals through the embedded pulse sequences. Finally, the system outputs and displays the super-resolution imaging results alongside the corresponding duplex contrast-enhanced imaging results.

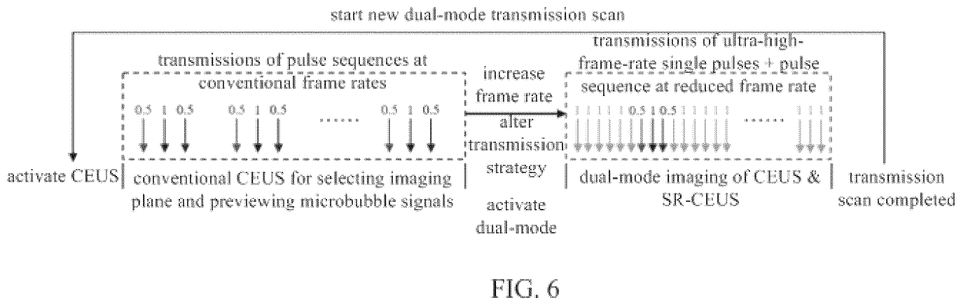

In summary, within the aforementioned flowchart, the overall transmission strategy is depicted in FIG. 6. Prior to activating the super-resolution mode, conventional contrast-enhanced imaging employs pulse sequences transmitted at a conventional frame rate to determine an imaging plane and assess microbubble signals. After activating the super-resolution mode (i.e., duplex imaging), the contrast-enhanced imaging involves emitting a combination of single pulses and sequence pulses, wherein: the single pulses with ultra-high frame rate generate echo signals used for super-resolution data acquisition and imaging processing in duplex imaging, while the interleaved sequence pulses produce echo signals utilized for conventional contrast-enhanced imaging in the duplex mode.

The above exemplarily illustrates the CEUS imaging method 100 according to embodiments of the present application. Based on the preceding description, the CEUS method 100 of this disclosure achieves the following: by transmitting an ultrasound pulse combinations comprising one or more single pulses and one or more pulse sequences to a target object injected with a contrast agent, the echo signals corresponding to the single pulses are utilized for SR-CEUS imaging, while the echo signals corresponding to the pulse sequences are used for real-time microbubble imaging. This method enables simultaneous achievement of ultra-high frame rate data acquisition to reduce super-resolution imaging data collection time and real-time visualization of microbubble dynamics. Consequently, doctors can not only observe microbubble behavior but also obtain super-resolution images to examine microvascular structures and other minute tissue conditions.

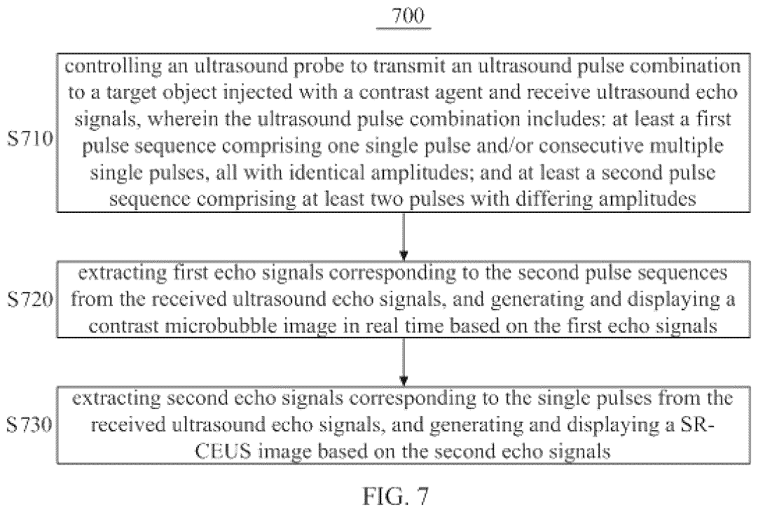

The following describes a CEUS imaging method 700 according to another embodiment of the present disclosure with reference to FIG. 7. As shown in FIG. 7, the CEUS imaging method 700 may comprise the following steps:

Step S710: controlling an ultrasound probe to transmit an ultrasound pulse combination to a target object injected with a contrast agent and receive ultrasound echo signals, wherein the ultrasound pulse combination includes: at least one first pulse sequence comprising one single pulse and/or a plurality of consecutive single pulses, all with identical amplitudes; and at least one second pulse sequence comprising at least two pulses with differing amplitudes.

Step S720: extracting first echo signals corresponding to the second pulse sequences from the received ultrasound echo signals, and generating and displaying a contrast microbubble image in real time based on the first echo signals.

Step S730: extracting second echo signals corresponding to the single pulses from the received ultrasound echo signals, and generating and displaying a SR-CEUS image based on the second echo signals.

The CEUS imaging method 700 according to some embodiments of the present application is substantially similar to the previously described CEUS imaging method 100 of the present application, with the following distinctions: in the CEUS imaging method 100, multiple ultrasound pulse combinations are transmitted, each combination comprising a plurality of consecutive single pulses (all having identical amplitudes) and one pulse sequence containing at least two pulses with different amplitudes; in contrast, the CEUS imaging method 700 transmits an ultrasound pulse combination comprising at least one first pulse sequence and at least one second pulse sequence, wherein the first pulse sequence includes one single pulse and/or a plurality of consecutive single pulses (the single pulses having identical amplitudes), while the second pulse sequence includes at least two pulses with different amplitudes. Thus, in the CEUS imaging method 700, one or more single pulses are also treated as a pulse sequence, except that the first pulse sequence containing the single pulses differs from the other pulse sequence (i.e., the second pulse sequence). Following principles similar to the CEUS imaging method 100, by transmitting an ultrasound pulse combination comprising a first pulse sequence (that includes one or more single pulses with identical amplitudes) and a second pulse sequence (that includes at least two pulses with different amplitudes) to a target object injected with a contrast agent, and utilizing echo signals corresponding to the first pulse sequence for SR-CEUS imaging and echo signals corresponding to the second pulse sequence for real-time microbubble imaging, the method 700 allows ultra-high frame rate data acquisition to reduce super-resolution imaging data collection time while maintaining real-time visualization of microbubble activity, enabling doctors to observe both microbubble behavior and super-resolution images of microvascular and other fine tissue structures.

In some embodiments of the present application, the ultrasound pulse combination comprises a plurality of first pulse sequences, wherein the pulse counts in all of the plurality of first pulse sequences are identical. In such embodiments, the ultrasound pulse combination includes multiple first pulse sequences, each of which comprises the same number of single pulses. This configuration enables uniform acquisition of echo signals corresponding to the single pulses and facilitates generation of more stable super-resolution images. This, however, is merely exemplary. In other embodiments, the ultrasound pulse combination comprises multiple first pulse sequences, wherein at least one first pulse sequence comprises a different number of pulses compared to other first pulse sequences. In such embodiments, the ultrasound pulse combination includes multiple first pulse sequences, but the number of pulses included in different first pulse sequences may vary.

The remaining aspects of the CEUS imaging method 700 are substantially similar to the corresponding components of the previously described method 100, and specific details are not reiterated here for conciseness, with only key operational features outlined.

In embodiments of the present disclosure, the single pulses have a first amplitude, and the amplitude of at least one pulse in the second pulse sequence is this first amplitude.

In some embodiments, the second pulse sequence includes three pulses, where the amplitude of one pulse equals the sum of the amplitudes of the other two.

In some embodiments, the first amplitude corresponds to the amplitude of a full-amplitude pulse, with one pulse in the three-pulse sequence having the full amplitude while the remaining two pulses have half-amplitude values.

In some embodiments, the second pulse sequence includes two pulses, where one pulse has the full amplitude and the other has half amplitude.

In some embodiments, the single pulses are transmitted to the target object at a single angle or multiple angles, and the pulses in the pulse sequences are similarly transmitted at a single angle or multiple angles.

In some embodiments, prior to controlling the ultrasound probe to transmit multiple ultrasound pulse combinations to the target object injected with the contrast agent, the method further includes: controlling the ultrasound probe to transmit one or more ultrasound pulse sequences to the target object and receive third echo signals; generating and displaying a third CEUS image based on the third echo signals; determining a ROI of the target object based on the third CEUS image; and controlling the ultrasound probe to transmit multiple ultrasound pulse combinations to the ROI of the target object.

The CEUS imaging method 700 according to embodiments of the present disclosure, as described above, achieves simultaneous ultra-high-frame-rate data acquisition and real-time microbubble visualization by transmitting an ultrasound pulse combination comprising a first pulse sequence (that includes one or more single pulses of identical amplitude) interleaved with a second pulse sequence (that includes at least two pulses of differing amplitudes) to a target object injected with a contrast agent, and utilizing echo signals corresponding to the first pulse sequence for SR-CEUS imaging and echo signals corresponding to the second pulse sequence for real-time microbubble imaging. This method enables concurrent reduction of super-resolution imaging data collection time through accelerated sampling and real-time display of microbubble dynamics during scanning, thus allowing doctors to both monitor live microbubble perfusion characteristics and obtain super-resolution images for detailed observation of microvascular structures and other microscopic tissue features.

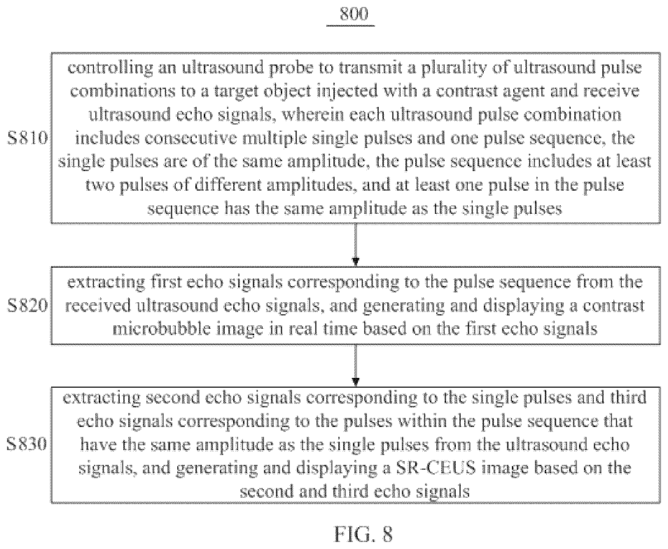

The following describes a CEUS imaging method 800 according to some further embodiments of the present disclosure with reference to FIG. 8. As shown in FIG. 8, the CEUS imaging method 800 may comprise the following steps:

Step S810: controlling an ultrasound probe to transmit multiple ultrasound pulse combinations to a target object injected with a contrast agent and receive ultrasound echo signals, wherein each ultrasound pulse combination includes a plurality of consecutive single pulses and one pulse sequence, the single pulses are of the same amplitude, the pulse sequence includes at least two pulses of differing amplitudes, and at least one pulse in the pulse sequence sharing the same amplitude as the single pulses.

Step S820: extracting first echo signals corresponding to the pulse sequence from the received ultrasound echo signals, and generating and displaying a contrast microbubble image in real time based on the first echo signals.

Step S830: extracting second echo signals corresponding to the single pulses and third echo signals corresponding to pulses within the pulse sequence that share the same amplitude as the single pulses from the ultrasound echo signals; and generating and displaying a SR-CEUS image based on the second and third echo signals.

The CEUS imaging method 800 according to embodiments of the present disclosure is substantially similar to the previously described method 100, with the following distinctions: in method 100, there is no restriction requiring that at least one pulse in the pulse sequence shares the same amplitude as the single pulses, and step S130 involves generating and displaying the SR-CEUS image based on the second echo signals corresponding to the single pulses from the ultrasound echo signals; in contrast, the CEUS imaging method 800 specifies that at least one pulse in the pulse sequence has the same amplitude as the single pulses, and step S830 involves extracting the second echo signals (corresponding to the single pulses) and the third echo signals (corresponding to the pulses in the pulse sequence that have the same amplitude value as the single pulses) from the ultrasound echo signals and generating and displaying a SR-CEUS image based on the second echo signals and the third echo signals. Accordingly, compared to method 100, the generation of the SR-CEUS image in method 800 is based not only on the echo signals corresponding to the single pulses but also incorporates the echo signals corresponding to the pulses in the pulse sequence that share the same amplitude as the single pulses. This enhancement significantly increases the data volume (frame rate) available for SR-CEUS imaging, thereby enabling the generation of higher-quality SR-CEUS images with improved spatial resolution. Additionally, this method reduces the number of pulses required for transmission. Other aspects of method 800 remain consistent with the corresponding details of method 100 described earlier; for conciseness, these are not reiterated here.

The CEUS imaging method 800 according to embodiments of the present disclosure, as described above, achieves enhanced performance by transmitting an ultrasound pulse combination comprising single pulses interleaved with pulse sequences (where at least one pulse in each pulse sequence shares the same amplitude as the single pulses) to a target object injected with a contrast agent, utilizing echo signals corresponding to the pulse sequences for real-time microbubble imaging, and utilizing echo signals corresponding to the single pulses and echo signals corresponding to the pulses in the pulse sequence that have the same amplitude as the single pulses for SR-CEUS imaging. This method enables ultra-high frame rate data acquisition to reduce super-resolution imaging data collection time, further enhances super-resolution image quality (via improved spatial resolution), and maintains real-time visualization of microbubble dynamics. Consequently, doctors can simultaneously observe microbubble perfusion and obtain super-resolution images for detailed evaluation of microvascular and other fine tissue structures.

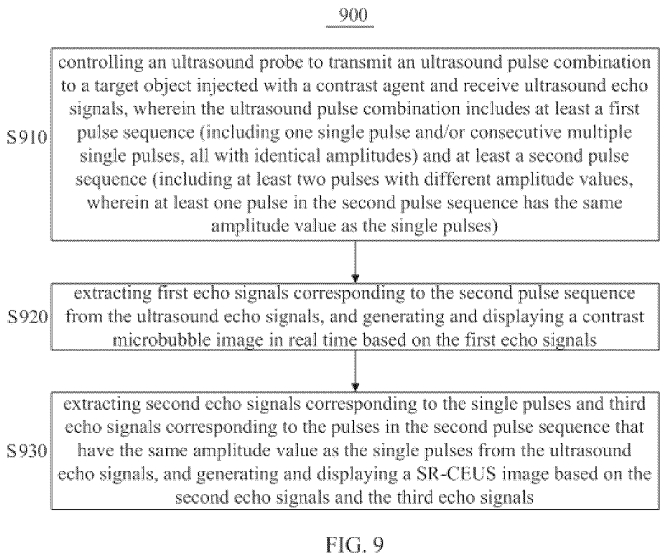

The following describes the CEUS imaging method 900 according to some embodiments of the present disclosure with reference to FIG. 9. As shown in FIG. 9, the CEUS imaging method 900 may include the following steps:

Step S910: controlling an ultrasound probe to transmit an ultrasound pulse combination to a target object injected with a contrast agent and receive ultrasound echo signals, wherein the ultrasound pulse combination includes at least a first pulse sequence (including one single pulse and/or a plurality of consecutive single pulses, all with identical amplitudes) and at least a second pulse sequence (including at least two pulses with different amplitude values, wherein at least one pulse in the second pulse sequence has the same amplitude value as the single pulses).

Step S920: extracting first echo signals corresponding to the second pulse sequence from the ultrasound echo signals, and generating and displaying a contrast microbubble image in real time based on the first echo signals.

Step S930: extracting second echo signals corresponding to the single pulses and third echo signals corresponding to the pulses in the second pulse sequence that have the same amplitude value as the single pulses from the ultrasound echo signals, and generating and displaying a SR-CEUS image based on the second echo signals and the third echo signals.

The CEUS imaging method 900 according to embodiments of the present disclosure is generally similar to the CEUS imaging method 800 described above. The difference therebetween lies in that: in the CEUS imaging method 800 of the embodiments, multiple ultrasound pulse combinations are transmitted, where each ultrasound pulse combination includes multiple consecutive single pulses and one pulse sequence, the single pulses are single pulses with the same amplitude value, and the pulse sequence includes at least two pulses with different amplitude values; in contrast, the ultrasound pulse combination transmitted in the CEUS imaging method 900 of the embodiments include at least one first pulse sequence and at least one second pulse sequence, where the first pulse sequence includes one single pulse and/or a plurality of consecutive single pulses (all being single pulses with the same amplitude value), and the second pulse sequence includes at least two pulses with different amplitude values. Thus, in the CEUS imaging method 900, one or more single pulses are also treated as part of pulse sequences, except that the first pulse sequence containing the single pulses differs from the other pulse sequence (i.e., the second pulse sequence). Other aspects of the CEUS imaging method 900 are similar to corresponding parts in the previously described CEUS imaging method 800. For conciseness, specific details will not be reiterated here.

Based on the above description, the CEUS imaging method 900 according to embodiments of the present disclosure transmits an ultrasound pulse combination comprising a first pulse sequence (including one or more single pulses with identical amplitude values) and a second pulse sequence (including at least two pulses with different amplitude values) to a target object injected with a contrast agent. Here, at least one pulse in the second pulse sequence shares the same amplitude value as the single pulses. The method utilizes echo signals corresponding to the second pulse sequence for real-time microbubble imaging, while employing echo signals from the single pulses and those corresponding to pulses in the second pulse sequence that match the amplitude of the single pulses for super-resolution contrast imaging. This method achieves ultra-high frame rate data acquisition to reduce super-resolution imaging data collection time while further enhancing the quality of super-resolution images. Additionally, it reduces the number of transmitted pulses and enables real-time visualization of microbubble dynamics. Consequently, doctors can not only observe microbubble behavior but also obtain super-resolution images to examine microvascular structures and other minute tissue details.

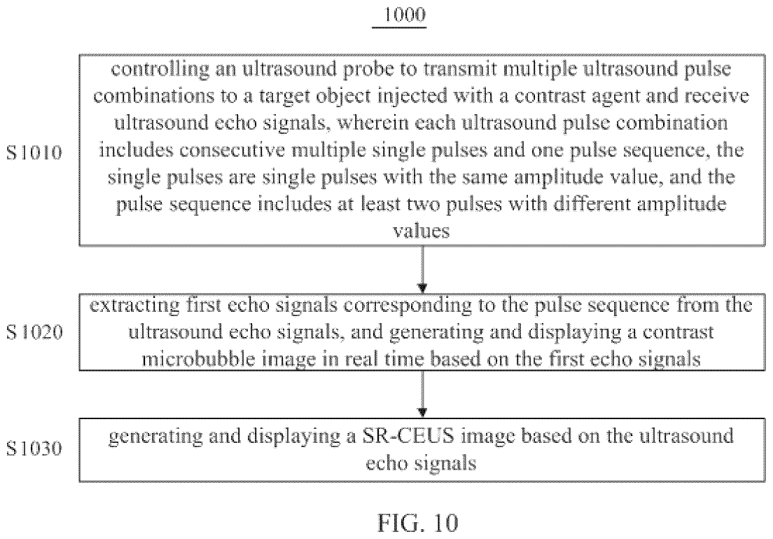

The following describes the CEUS imaging method 1000 according to yet some embodiments of the present disclosure with reference to FIG. 10. As shown in FIG. 10, the CEUS imaging method 1000 may include the following steps:

Step S1010: controlling an ultrasound probe to transmit multiple ultrasound pulse combinations to a target object injected with a contrast agent and receive ultrasound echo signals, wherein each ultrasound pulse combination includes a plurality of consecutive single pulses and one pulse sequence, the single pulses are single pulses with the same amplitude value, and the pulse sequence includes at least two pulses with different amplitude values.

Step S1020: extracting first echo signals corresponding to the pulse sequence from the ultrasound echo signals, and generating and displaying a contrast microbubble image in real time based on the first echo signals.

Step S1030: generating and displaying a SR-CEUS image based on the ultrasound echo signals.

The CEUS imaging method 1000 according to embodiments of the present disclosure is substantially similar to the previously described CEUS imaging method 100, with the following distinctions: in method 100, step S130 involves generating and displaying the SR-CEUS image based on the second echo signals that correspond to the single pulses extracted from the ultrasound echo signals; in contrast, method 1000 in step S1030 generates and displays the SR-CEUS image using all the ultrasound echo signals. Consequently, compared to method 100, the generation of the super-resolution contrast image in method 1000 relies not only on echo signals from the single pulses but also incorporates echo signals from the pulse sequences. This enhancement increases the data volume (frame rate) available for super-resolution imaging, thereby producing higher-quality results (with improved spatial resolution) and reducing the number of pulses required for transmission. Other aspects of the CEUS imaging method 1000 align with corresponding parts of the CEUS imaging method 100 described earlier. For brevity, specific details are not repeated here.

Based on the above description, the CEUS imaging method 1000 according to embodiments of the present disclosure transmits an ultrasound pulse combination comprising single pulses and pulse sequences to a target object injected with a contrast agent. The method utilizes echo signals corresponding to the pulse sequences for real-time microbubble imaging, while employing both echo signals from the single pulses and those from the pulse sequences for super-resolution contrast imaging. This approach achieves ultra-high frame rate data acquisition to reduce super-resolution imaging data collection time while further enhancing the quality of super-resolution images. It also reduces the number of transmitted pulses and enables real-time visualization of microbubble dynamics. As a result, doctors can not only monitor microbubble behavior but also obtain super-resolution images to observe microvascular structures and other minute tissue details.

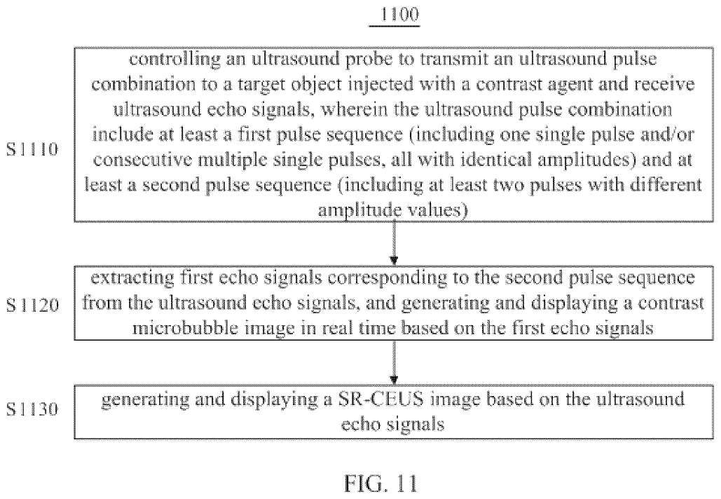

The following describes the CEUS imaging method 1100 according to yet other embodiments of the present disclosure with reference to FIG. 11. As shown in FIG. 11, the CEUS imaging method 1100 may include the following steps:

Step S1110: controlling an ultrasound probe to transmit an ultrasound pulse combination to a target object injected with a contrast agent and receive ultrasound echo signals, wherein the ultrasound pulse combination include at least a first pulse sequence (including one single pulse and/or a plurality of consecutive single pulses, all with identical amplitudes) and at least a second pulse sequence (including at least two pulses with different amplitude values).

Step S1120: extracting first echo signals corresponding to the second pulse sequence from the ultrasound echo signals, and generating and displaying a contrast microbubble image in real time based on the first echo signals.

Step S1130: generating and displaying a SR-CEUS image based on the ultrasound echo signals.

The CEUS imaging method 1100 according to embodiments of the present disclosure is generally similar to the previously described CEUS imaging method 1000. The difference therebetween lies in that: in the CEUS imaging method 1000 of the embodiments, multiple ultrasound pulse combinations are transmitted, each including multiple consecutive single pulses and one pulse sequence, the single pulses are single pulses with the same amplitude value, and the pulse sequence includes at least two pulses with different amplitude values; in contrast, the ultrasound pulse combination transmitted in the CEUS imaging method 1100 of the embodiments include at least one first pulse sequence and at least one second pulse sequence, the first pulse sequence includes one single pulse and/or a plurality of consecutive single pulses (all being single pulses with the same amplitude value), while the second pulse sequence includes at least two pulses with different amplitude values. Thus, in the CEUS imaging method 1100, one or more single pulses are also treated as part of pulse sequences, except that the first pulse sequence containing the single pulses differs from the other pulse sequence (i.e., the second pulse sequence). Other aspects of the CEUS imaging method 1100 align with corresponding parts of the CEUS imaging method 1000 described earlier. For conciseness, specific details are not reiterated here.

Based on the above description, the CEUS imaging method 1100 according to embodiments of the present disclosure transmits the ultrasound pulse combination comprising a first pulse sequence (including one or more single pulses with identical amplitude values) and a second pulse sequence (including at least two pulses with different amplitude values) to a target object injected with a contrast agent. The method utilizes echo signals corresponding to the second pulse sequence for real-time microbubble imaging, while employing echo signals from the first pulse sequence (single pulses) and the second pulse sequence for SR-CUES imaging. This method achieves ultra-high frame rate data acquisition to reduce super-resolution imaging data collection time while further improving the quality of super-resolution images. Additionally, it reduces the number of transmitted pulses and enables real-time visualization of microbubble dynamics. As a result, doctors can not only observe microbubble behavior but also obtain super-resolution images to examine microvascular structures and other minute tissue details.

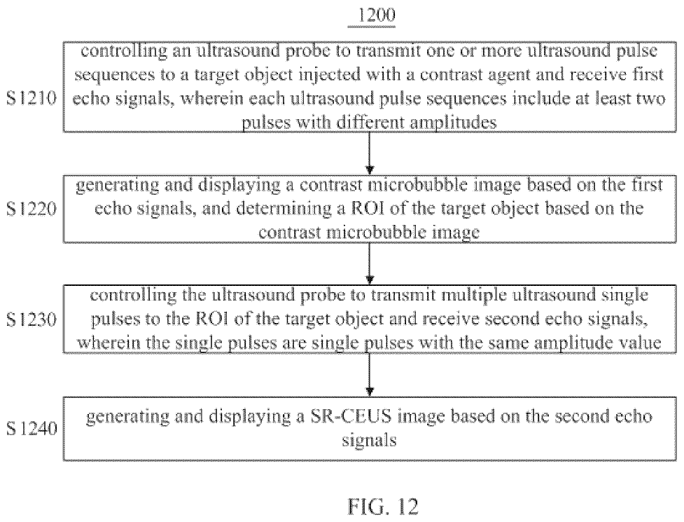

The following describes the CEUS imaging method 1200 according to yet other embodiments of the present disclosure with reference to FIG. 12. As shown in FIG. 12, the CEUS imaging method 1200 may include the following steps:

Step S1210: controlling an ultrasound probe to transmit one or more ultrasound pulse sequences to a target object injected with a contrast agent and receive first echo signals, wherein each ultrasound pulse sequences include at least two pulses with different amplitudes.

Step S1220: generating and displaying a contrast microbubble image based on the first echo signals, and determining a ROI of the target object based on the contrast microbubble image.

Step S1230: controlling the ultrasound probe to transmit multiple ultrasound single pulses to the ROI of the target object and receive second echo signals, wherein the single pulses are single pulses with the same amplitude value.

Step S1240: generating and displaying a SR-CEUS image based on the second echo signals.

The CEUS imaging method 1200 according to embodiments of the present disclosure generates and displays a contrast microbubble image by transmitting ultrasound pulse sequences to a target object injected with a contrast agent, thereby determining the target object's region of interest. Subsequently, multiple ultrasound single pulses are transmitted to the ROI to generate and display a SR-CEUS image. As previously described in connection with FIG. 1, compared to conventional super-resolution imaging using pulse sequences transmitted at regular frame rates, the use of single pulses enhances the frame rate, thereby reducing data collection time required for super-resolution imaging. Thus, method 1200 enables super-resolution contrast imaging with single pulses transmitted at a frame rate higher than conventional rates, significantly shortening data acquisition time. Additionally, since pulse sequences are first transmitted for conventional contrast imaging before single pulses, users can review microbubble dynamics prior to observing the super-resolution images.

Regarding the single pulses and pulse sequences in the CEUS imaging method 1200, their details are similar to those described earlier in connection with FIG. 1 and will not be reiterated here, with only key operational features outlined.

In embodiments of the present disclosure, the amplitude value of the single pulses is a first amplitude value, and at least one pulse in the pulse sequences shares this first amplitude value.

In embodiments, each ultrasound pulse sequence includes three pulses, where one pulse has an amplitude value equal to the sum of the amplitudes of the other two pulses.

In embodiments, the first amplitude value corresponds to a full-amplitude pulse, wherein one of the three pulses in the sequence has this full-amplitude value, while each of the remaining two pulses has a half-amplitude value.

In embodiments, an ultrasound pulse sequence includes two pulses, where one pulse has a full-amplitude value and the other has a half-amplitude value.

In embodiments, pulses in the ultrasound pulse sequences are transmitted to the target object using single-angle or multi-angle transmissions. Similarly, each single pulse is transmitted to the target object using single-angle or multi-angle transmissions.

Based on the above description, the CEUS imaging method 1200 according to embodiments of the present disclosure enables SR-CEUS imaging by transmitting single pulses at a frame rate higher than conventional rates, thereby significantly reducing data acquisition time. Additionally, since pulse sequences are transmitted for conventional contrast imaging prior to single pulse transmission, users can review microbubble dynamics before accessing super-resolution images. This sequential workflow ensures that doctors can first assess microbubble behavior and subsequently obtain detailed super-resolution images of microvascular structures and other fine tissue features.

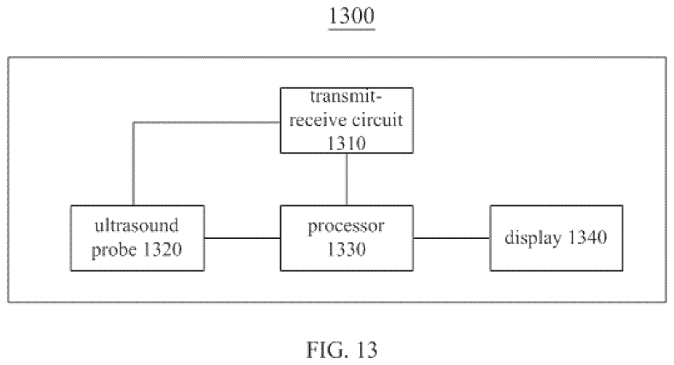

The following describes an ultrasound imaging apparatus according to another aspect of the present disclosure with reference to FIG. 13. FIG. 13 illustrates a schematic structural block diagram of the ultrasound imaging apparatus 1300 according to some embodiments of the present disclosure. As shown in FIG. 13, the ultrasound imaging apparatus 1300 may include a transmit-receive circuit 1310, an ultrasound probe 1320, a processor 1330, and a display 1340. Specifically: the transmit-receive circuit 1310 is configured to control the ultrasound probe 1320 to transmit ultrasound waves to a target object, receive echoes of the ultrasound waves, and extract ultrasound echo signals from the echoes; the processor 1330 is configured to control the transmit-receive circuit and execute the CEUS imaging methods 100, 700, 800, 900, 1000, 1100, or 1200 described in the preceding embodiments of the present disclosure to generate a contrast microbubble image and a SR-CUES image; and the display 1340 is configured to display the contrast microbubble image and the SR-CUES image. The CEUS imaging methods 100, 700, 800, 900, 1000, 1100, and 1200 according to the embodiments of the present disclosure have been described in detail earlier. Those skilled in the art may understand the structure and operation of the ultrasound imaging apparatus 1300 based on the preceding descriptions. For brevity, further details are omitted here.

Furthermore, according to embodiments of the present disclosure, a storage medium is provided. Program instructions are stored on the storage medium, which, when executed by a computer or processor, are configured to perform the corresponding steps of the CEUS imaging methods described in the embodiments of the present disclosure. The storage medium may include, for example, a smartphone memory card, a tablet storage component, a personal computer hard disk, read-only Memory (ROM), erasable programmable read-only memory (EPROM), portable compact disc read-only memory (CD-ROM), USB storage devices, or any combination of the aforementioned storage media. The computer-readable storage medium may comprise any combination of one or more computer-readable storage media.

Furthermore, according to embodiments of the present disclosure, a computer program is provided. The computer program may be stored on cloud-based or local storage media. When the computer program is executed by a computer or processor, it is configured to perform the corresponding steps of the CEUS imaging methods described in the embodiments of the present disclosure.

Based on the above description, the CEUS imaging methods and apparatus according to embodiments of the present disclosure enable SR-CEUS imaging by transmitting single pulses at a frame rate higher than conventional rates, thereby reducing data acquisition time.

While exemplary embodiments have been described herein with reference to the accompanying drawings, it is to be understood that these embodiments are illustrative and not intended to limit the scope of the present disclosure. Those skilled in the art may make various variations and modifications without departing from the scope and spirit of the present disclosure. All such variations and modifications are intended to fall within the scope of the present disclosure as defined by the appended claims.

Those skilled in the art will recognize that the units and algorithmic steps described in the embodiments disclosed herein can be implemented through electronic hardware, or a combination of computer software and electronic hardware. Whether these functions are executed via hardware or software depends on the specific application and design constraints of the technical solution. Professionals may employ different methods to implement the described functionalities for each specific application, but such implementations should not be interpreted as exceeding the scope of the present disclosure.

In the embodiments provided by the present disclosure, it should be understood that the disclosed apparatus and methods may be implemented in alternative ways. For example, the device embodiments described above are merely illustrative. The division of units, for instance, represents a logical functional division and may differ in practical implementations. Multiple units or components may be combined or integrated into another device, or certain features may be omitted or excluded from execution.

In the specification disclosed herein, numerous specific details are described. However, it is to be understood that the embodiments of the present disclosure may be practiced without these specific details. In some instances, well-known methods, structures, and techniques have not been shown in detail to avoid obscuring the understanding of this specification.

Similarly, it should be understood that, to streamline the present disclosure and aid in understanding one or more of its inventive aspects, the features of the disclosure are sometimes grouped together into a single embodiment, figure, or description thereof in the context of describing exemplary embodiments. However, this method of disclosure should not be interpreted as reflecting an intention that the claimed disclosure requires more features than those expressly recited in each claim. Rather, as reflected in the corresponding claims, the inventive point lies in addressing the relevant technical problem using features fewer than all those disclosed in any single embodiment. Accordingly, the claims following the Detailed Description are hereby expressly incorporated into the Detailed Description, with each claim standing on its own as a separate embodiment of the present disclosure.

Those skilled in the art will understand that, unless features are mutually exclusive, any combination of all features disclosed in this specification (including the accompanying claims, abstract, and drawings), as well as all processes or units of any method or apparatus disclosed herein, may be utilized. Unless expressly stated otherwise, each feature disclosed in this specification (including the accompanying claims, abstract, and drawings) may be replaced by an alternative feature serving the same, equivalent, or similar purpose.

Additionally, those skilled in the art will understand that while some embodiments described herein include certain features present in other embodiments while excluding others, the combination of features from different embodiments falls within the scope of the present disclosure and may form distinct embodiments. For example, in the claims, any of the claimed embodiments may be used in any combination.

The various component embodiments of the present disclosure may be implemented in hardware, as software modules running on one or more processors, or as a combination thereof. Those skilled in the art will recognize that microprocessors or digital signal processors (DSPs) may be employed in practice to realize some or all functionalities of the modules in the item analysis apparatus according to embodiments of the present disclosure. The present disclosure may also be implemented as apparatus programs (e.g., computer programs and computer program products) configured to execute part or all of the methods described herein. Such programs implementing the disclosure may be stored on computer-readable media or exist in the form of one or more signals. Such signals may be downloaded from internet websites, provided on carrier signals, or delivered in any other form.

It should be noted that the above embodiments illustrate the present disclosure rather than limit it, and those skilled in the art may devise alternative embodiments without departing from the scope of the appended claims. In the claims, any reference signs placed between parentheses shall not be construed as limiting the claims. The term “comprising” does not exclude the presence of elements or steps not listed in the claims. The use of the word “a” or “an” preceding an element does not exclude the existence of multiple such elements. The present disclosure may be implemented using hardware comprising several distinct components or an appropriately programmed computer. In unit claims enumerating multiple means, several of these means may be embodied by the same hardware item. The use of terms such as “first,” “second,” and “third” does not imply any order. These terms are to be interpreted as designations.

The above descriptions are merely specific implementations of the present disclosure or explanations thereof. The scope of protection of the present disclosure is not limited thereto. Any modifications or substitutions that could readily be conceived by those skilled in the art within the technical scope disclosed by the present disclosure shall fall within the scope of protection of the present disclosure. The scope of protection of the present disclosure shall be determined by the scope defined in the claims.

What is claimed is:

1. A contrast-enhanced ultrasound imaging method, comprising:
controlling an ultrasound probe to transmit a plurality of ultrasound pulse combinations to a target object injected with a contrast agent and receive ultrasound echo signals, wherein each of the ultrasound pulse combinations comprises a plurality of consecutive single pulses and one pulse sequence, the single pulses are of a same amplitude, and the pulse sequence comprises at least two pulses with different amplitudes;
acquiring first echo signals corresponding to the pulse sequence from the ultrasound echo signals, generating and displaying a contrast microbubble image in real time based on the first echo signals; and
acquiring second echo signals corresponding to the single pulses from the ultrasound echo signals, and generating and displaying a super-resolution contrast-enhanced ultrasound (SR-CEUS) image based on the second echo signals.

2. The method according to claim 1, wherein
the amplitude of the single pulses is a first amplitude, and
at least one pulse in the pulse sequence has an amplitude equal to the first amplitude.

3. The method according to claim 2, wherein
the pulse sequence comprises three pulses, wherein one of the three pulses has an amplitude equal to a sum of amplitudes of remaining two pulses.

4. The method according to claim 1, wherein
amplitudes of the pulses in the pulse sequence are symmetrically distributed.

5. The method according to claim 1, wherein
the pulse sequence comprises two pulses, wherein one of the two pulses has an amplitude of a full-amplitude pulse, and the other pulse has an amplitude of a half-amplitude pulse.

6. The method according to claim 1, wherein
the single pulses are transmitted to the target object at a single angle or a plurality of angles; and
the pulses in the pulse sequence are transmitted to the target object at the single angle or the plurality of angles.

7. The method according to claim 1, wherein
before controlling an ultrasound probe to transmit a plurality of ultrasound pulse combinations to a target object injected with a contrast agent, the method further comprises:
controlling the ultrasound probe to transmit one or more ultrasound pulse sequences to the target object and receive third echo signals;
generating and displaying a third contrast-enhanced ultrasound (CEUS) image based on the third echo signals;
acquiring a region of interest of the target object based on the third CEUS image; and
controlling the ultrasound probe to transmit the plurality of ultrasound pulse combinations to the region of interest of the target object.

8. The method according to claim 7, wherein
the one or more ultrasound pulse sequences are identical to the pulse sequence in the ultrasound pulse combinations.

9. The method according to claim 1, wherein
generating and displaying a SR-CEUS image is further based on the first echo signals.

10. The method according to claim 1, wherein
an imaging region corresponding to the first echo signals is identical to an imaging region corresponding to the second echo signals.

11. A contrast-enhanced ultrasound imaging method, comprising:
controlling an ultrasound probe to transmit an ultrasound pulse combination to a target object injected with a contrast agent and receive ultrasound echo signals, wherein the ultrasound pulse combination comprises at least a first pulse sequence and at least a second pulse sequence, the first pulse sequence comprises one single pulse and/or a plurality of consecutive single pulses, the single pulses are of a same amplitude, and the second pulse sequence comprises at least two pulses with different amplitudes;
acquiring first echo signals corresponding to the second pulse sequence from the ultrasound echo signals, generating and displaying a contrast microbubble image in real time based on the first echo signals; and
acquiring second echo signals corresponding to the single pulses from the ultrasound echo signals, and generating and displaying a super-resolution contrast-enhanced ultrasound (SR-CEUS) image based on the second echo signals.

12. The method according to claim 11, wherein
the ultrasound pulse combination comprises a plurality of first pulse sequences, wherein each of the plurality of first pulse sequences comprises a same number of pulses.

13. The method according to claim 11, wherein
the ultrasound pulse combination comprises a plurality of first pulse sequences, wherein at least one of the plurality of first pulse sequences has a number of pulses different from that of remaining of the plurality of first pulse sequences.

14. The method according to claim 11, wherein
the amplitude of the single pulses is a first amplitude; and
at least one pulse in the second pulse sequence has an amplitude equal to the first amplitude.

15. The method according to claim 14, wherein
the second pulse sequence comprises three pulses, and one of the three pulses has an amplitude equal to a sum of amplitudes of remaining two pulses.

16. The method according to claim 11, wherein
the second pulse sequence comprises two pulses, wherein one of the two pulses has an amplitude of a full-amplitude pulse, and the other pulse has an amplitude of a half-amplitude pulse.

17. A contrast-enhanced ultrasound imaging method, comprising:
controlling an ultrasound probe to transmit one or more ultrasound pulse sequences to a target object injected with a contrast agent and receive first echo signals, wherein the one or more ultrasound pulse sequences comprise at least two pulses with different amplitudes;
generating and displaying a contrast microbubble image based on the first echo signals, and acquiring a region of interest of the target object based on the contrast microbubble image;
controlling the ultrasound probe to transmit a plurality of ultrasound single pulses to the region of interest of the target object and receive second echo signals, wherein the single pulses are of a same amplitude; and
generating and displaying a super-resolution contrast-enhanced ultrasound (SR-CEUS) image based on the second echo signals.

18. The method according to claim 17, wherein
the amplitude of the single pulses is a first amplitude; and
at least one pulse in the one or more pulse sequences has an amplitude equal to the first amplitude.

19. The method according to claim 18, wherein
each ultrasound pulse sequence comprises three pulses, wherein one of the three pulses has an amplitude equal to a sum of amplitudes of remaining two pulses.

20. The method according to claim 17, wherein
each ultrasound pulse sequence comprises two pulses, wherein one of the two pulses has an amplitude of a full-amplitude pulse, and the other pulse has an amplitude of a half-amplitude pulse.
